(19) United States
(12) Patent Application Publication
(10) Pub. No.: US 2003/0004418 A1
(43) Pub. Date: Jan. 2, 2003

(54) Apparatus and method for ratiometric quantitation of elicited autofluorescence of the eye

(76) Inventor: Alan D. Marmorstein, Shaker Heights, OH (US)

Correspondence Address: BENESCH, FRIEDLANDER, COPLAN & ARONOFF LLP, ATTN: IP DEPARTMENT DOCKET CLERK, 2300 BP TOWER, 200 PUBLIC SQUARE, CLEVELAND, OH, 44114, US

(21) Appl. No.: 10/159,248

(22) Filed: May 29, 2002

Related U.S. Application Data
(60) Provisional application No. 60/298,548, filed on Jun. 15, 2001.

(51) Int. Cl. A61B 6/00

(52) U.S. Cl. 600/475; 600/477; 356/317

(57) ABSTRACT

An optical scanning spectroscopic apparatus includes a light source and optics to direct light into a region of interest. Means for detecting autofluorescent emissions at a plurality of detectably distinct wavelengths are also provided. A processor receives data corresponding to the autofluorescence and compares the data to a control data set. The light source may alternately include any source of suitable light such as an arc lamp, a laser, or a pulsed laser each controlled to produce a defined wavelength. The comparison of autoflourescent emissions collected at different wavelengths is claimed as a means for diagnosing various retinal diseases.

### CROSS REFERENCES TO RELATED APPLICATIONS

This application claims benefit of U.S. Provisional Application No. 60/298,548, filed Jun. 15, 2001.

### BACKGROUND OF THE INVENTION

The present invention relates generally to the detection of retinal conditions and characteristics and more particularly to a method and apparatus for the early detection of retinal degenerative diseases using autofluorescence spectroscopy.

Age-related macular degeneration (AMD) is the leading cause of blindness in the western world affecting nearly 30% of those over the age of 75. AMD alters the quality of life of those affected by causing a debilitating loss of central vision. Clinically, the disease is characterized by an increase in macular drusen, retinal pigmented epithelium (RPE) mottling or areas of geographic atrophy, and in some cases by choroidal neovasculatrization. Histopathologically, AMD is characterized by photoreceptor cell loss, accumulation of drusen, abnormal thickening of Bruch's membrane, confluent drusen, basal laminar deposits, and deposits within Bruch's membrane. In late stages, calcification of Bruch's membrane, and RPE and retinal atrophy are also observed. While a relationship between lipofuscin content of the RPE and AMD has been suggested, no quantitative analysis has fully addressed the relationship. Despite the above characteristics, extra-macular drusen and accumulation of lipofuscin can be found in nearly all eyes increasing with age.

Lipofuscin is a ubiquitous material present in granules in the RPE cell with a characteristic UV excitable orange fluorescence, which is accounted for in part by A2E, an adduct of vitamin A and phosphatidylethanolamine. In Stargardt's disease a relationship between lipofuscin accumulation and retinal degeneration is strongly supported by studies of the abcr knockout mouse as well as the fundus autofluorescence measurements of Delori and co-workers, and more recently by von Ruckman and co-workers. The recent development of the confocal scanning laser ophthalmoscope (cLSO) has greatly facilitated the study of fundus autofluorescence in humans. Studies using the cLSO and other means of measuring fundus autofluorescence have been for the most part limited to inherited maculopathies like Stargardt's, in which the role of lipofuscin is better established, or on patients with AMD who are already exhibiting areas of geographic atrophy. These studies have focused primarily on fluorescent emissions that are presumed to represent RPE associated lipofuscin. While some studies suggest that fundus autofluorescence may be elevated in areas peripheral to regions of geographic atrophy, or in advance of atrophy, fundus autofluorescence measurements have not gained wide acceptance as a diagnostic tool, and the connection between lipofuscin and AMD remains tenuous.

Lipofuscin granules in the RPE are not the only autofluorescent entities in the posterior of the eye. Autofluorescence of Bruch's membrane has also been casually reported, though the spectrum has never been characterized. More recently it has been observed in a systematic study of fundus autofluorescence, that the spectrum in regions with drusen is shifted toward shorter wavelengths.

In order to examine the posterior portion of a subject's eye, several non-invasive techniques have been developed. In this regard, the simplest technique for examining the posterior portions of a subject's eye is fundus photography. Fundus photography illuminates a subject's eye with a flash of white light, and then detects the reflected light returning from the subject's eye, using photographic film or a digital camera. A fundus photo typically contains an accurate reflection of a portion of the light from the retinal and choroidal vessels, as well as the reflection and scattering of portions of the light from other features of the posterior of the subject's eye. Fundus photography typically does not spectrally separate the light that returns from the subject's eye.

In 1979 the scanning laser opthalmoscope was introduced. This device increased the resolution of the fundus camera and improved over traditional fundus photography by permitting depth measurements of various features of the posterior pole (ie. optic disc). For a general description of scanning laser opthalmoscopes, see Noninvasive Diagnostic Techniques in Ophthalmology, Barry R. Masters, editor, Chapter 22, Scanning Laser Ophthalmoscope, by Robert H. Webb, Springer-Verlag, N.Y. (1990). Conventional scanning laser opthalmoscopes have a single laser source. The scanning laser opthalmoscope scans the laser signals emitted by the laser source in a predetermined pattern across posterior portions of a subject's eye to thereby define a frame having a number of scan lines. Since a single laser is employed, the resulting image will only provide information relating to reflection of the exciting light, or of the fluorescence excited at the one particular wavelength. Modification of the scanning laser opthalmoscope to incorporate additional laser lines has typically been applied to generating a color fundus photo similar to that obtained by traditional fundus photography, but with a higher degree of resolution.

Another application of the scanning laser opthalmoscope is the imaging of fundus autofluorescence. Though fundus autofluorescence imaging has some demonstrated diagnostic value for several inherited maculopathies, it has not become a standardized test for diagnosis of any disease. There is no evidence to suggest that fundus autofluorescence imaging as it is currently practiced could reveal basal laminar deposits, or that it could serve as an early diagnostic test for AMD.

While traditional fundus photography and scanning laser opthalmoscope scans have utility, a new technique that overcomes the above-noted drawbacks is needed.

### SUMMARY OF THE INVENTION

In accordance with one embodiment of the present invention, a method includes receiving a first auto-fluorescent emission from an identifiable area of a specimen, and receiving a second auto-fluorescent emission from the identifiable area of the specimen, the first emission and second emission comprising ranges of wavelengths. Characteristics of the first and second auto-fluorescent emissions are compared and selected comparisons indicate disease of the specimen.

In accordance with another embodiment of the present invention, a system for evaluating a specimen includes an excitation source for exciting the specimen, a detector for detecting fluorescence from the specimen in response to the excitation, and a processor for processing data received from the detector. The processor includes a first input in data communication with a first detector element providing data indicative of a first fluorescence in a first wavelength band from an identifiable area of the specimen. The processor also includes a second input in data communication with a second detector element providing data indicative of a second fluorescence in a second wavelength band from the identifiable area of the specimen. The processor also includes a comparator that compares the data indicative of the first and second fluorescence, where selected comparisons indicate disease of the specimen.

For the purposes of this invention the term autoflourescence shall be construed as to encompass all fluorescent emissions that can be excited by light from any anatomically or histologically identifiable regions of a specimen without the addition of fluorescent chemical compounds that are not present in the eye during normal function.

In accordance with another embodiment of the present invention, a method for diagnosing and prognosticating retinal characteristics comprises: emitting light of a predetermined wavelength into a target area of the eye with the purpose of exciting autofluorescence from said target area of the eye; detecting autofluorescence excited at several different wavelengths by using beamsplitters, dichroic mirrors, and multiple detectors to separate different spectral components of the emission from a target area of the eye within the target area in response to illumination by the light; calculating a ratio of the autofluorescence intensity of the signals obtained against each other; and comparing the ratio to a predetermined data set to identify retinal characteristics and potential disease. The determinable retinal characteristics include AMD, macular holes, retinal defect, retinal disease and the like.

In accordance with another embodiment of the present invention, an optical scanning spectroscopic apparatus comprises: a light source for excitation of fluorescence including visible light for single photon excitation of fluorescence or a pulsed infrared light source to elicit multi-photon fluorescence. A means for detecting auto fluorescence comprising a plurality of detectably distinct wavelengths responsive to the excitation. A processor means for processing the intensity and plotting the intensities to X-Y coordinates using as a reference a reflected light image of the fundus, and then to compare the intensity to predetermined thresholds derived from a control data set. In one aspect, the device uses a beam splitter to allow for the simultaneous collection of emitted light at two different wavelengths. This preserves additional resolution by compensating for movement of the subject between flashes that would be required to collect emitted fluorescence using a single detector in series.

The method and apparatus may comprise the use of a light source that is an arc lamp with suitable narrow bandpass filter so as to define a specific wavelength to be used for fluorescence excitation, or a laser of a defined wavelength in the visible spectrum (400-750 nm), or a pulse laser to emit signals at a wavelength between 690 and 900 nm should muiltiphoton elicited fluorescence be desired. Furthermore, target areas of the eye include the neurosensory retina, Bruch's membrane, retinal pigment epithelium, and choroid. Fluorescence emissions are elicited specifically from lipofuscin granules within the RPE, or compounds present in Bruch's membrane or various sub-retinal pigment epithelium deposits as defined below and in Marmorstein et al., (IOVS, in Press).

The present invention provides a retinal disease diagnostic/prognostic method that utilizes the individual contributions of drusen, Bruch's membrane, and RPE lipofuscin of retinal autofluorescence by taking advantage of recent developments in confocal microscopy that allow the collection of emission spectra from X-Y scans of tissue sections.

Utilizing a laser scanning confocal microscope with a spectrophotometric detector it is shown that Bruch's membrane and drusen have overlapping spectra that are excited by UV and blue light with fluorescent emissions in the blue/green spectrum. Furthermore, the present invention demonstrates that this fluorescence is increased relative to lipofuscin fluorescence in eyes from donors with AMD relative to age matched controls. The present invention identifies the distinct spectra that allow a quantitative aspect of the measurement of fundus autofluorescence. Specifically, the quantitative measurement aspect is the ability to perform ratiometric measurements as an indicator of retinal characteristics.

The present invention uses a unique spectrum of autofluorescence that is elicited from Bruch's membrane and drusen in the eye when excited with UV (364 nm) illumination. The spectrum results in the emission of blue light from Bruch's membrane and drusen with a maximum intensity at 485 nm+5 nm. The intensity of this emission was found to be greatly enhanced relative to the 555 nm+5 nm emission of RPE associated lipofuscin. The identification of this 485 nm emission allows the implementation of a diagnostic criteria whereby the ratio of fluorescence emissions derived from some region of the peak centered at 485 nm versus the intensity measured at a defined region of the peak elicited at 555 nm are used as a quantitative measure. As used herein, the term drusen is defined to include any pathologic deposit located within Bruch's membrane or between Bruch's membrane and the RPE.

In another aspect of the present invention, visible light with a wavelength between 400 and 490 nm excites emissions from Bruch's membrane, drusen, and RPE/lipofuscin, and reduces the difficulties and dangers of using UV light

In another aspect of the present invention, a pulsed infrared light excites emissions from Bruch's membrane, drusen and RPE/lipofuscin and reduces the difficulties and dangers of utilizing UV or blue light illumination for fundus autofluorescence measurement.

### BRIEF DESCRIPTION OF FIGURES

The invention may take form in various parts and arrangements of parts, and in various steps and arrangements of steps. The drawings are only for purposes of illustrating the preferred embodiments and are not to be construed as limiting the invention.

FIG. 1 illustrates a simplified system diagram suitable to practice the invention;

FIG. 2 illustrates a simplified system diagram suitable to practice an alternate embodiment of the invention;

FIG. 3A illustrates a typical field from a control specimen obtained using differential interference contrast microscopy;

FIG. 3B is a typical field from a specimen known to be afflicted with age-related macular degeneration (AMD) obtained using differential interference contrast microscopy;

FIGS. 4A and 4C are graphs representative of spectra obtained from a control specimen similar to that shown in FIG. 3A using 633 nm [A] or 568 nm [C] excitation wavelengths;

FIGS. 4B and 4D are graphs representative of spectra taken from a specimen known to be afflicted with age-related macular degeneration (AMD) similar to that shown in FIG. 3B using 633 nm [B] or 568 nm [D] excitation wavelengths;

FIGS. 5A and 5B are graphs representative of spectra obtained from a control (A) specimen or a specimen known to have AMD (B) similar to those shown in FIG. 1 using a 488 nm excitation wavelength;

FIGS. 6A and 6B are graphs representative of spectra obtained from a control (A) specimen or a specimen known to have AMD (B) similar to those shown in FIG. 1 using a 364 nm excitation wavelength;

### DETAILED DESCRIPTION OF THE PREFERRED EMBODIMENTS

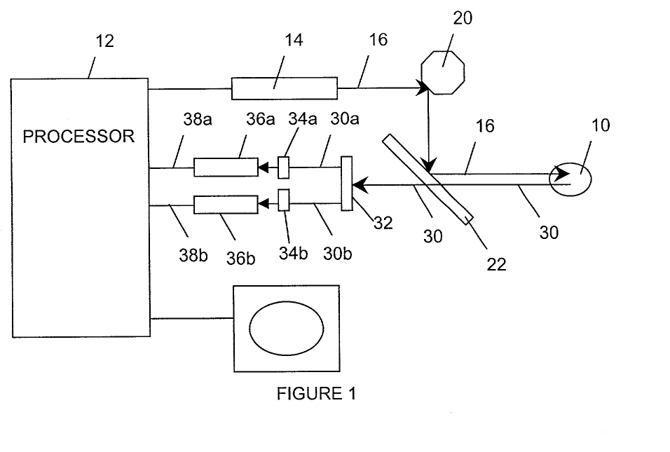

With reference now to FIG. 1, a system is shown for evaluating a specimen which suitably practices the present invention. A specimen 10 such as an eye, is placed relative to the system under control of a processor 12. The processor 12 controls an excitation source 14 such as an arc lamp or a laser which generates light 16. In a laser based system, the beam of light 16 is directed at a scan head 20 that acts to scan the light 16 in a defined path. The scanning light beam 16 is then directed into other optics 22 and focussed on the specimen 10. Those skilled in the art will appreciate that while the optics 22 are simply illustrated, actual optics associated with a system for fluorescence imaging are considerably more complex. Furthermore, lasers of any available wavelength can be used to provide different excitation wavelengths. Alternatively, the laser can be substituted with an arc lamp (i.e. Xenon or Mercury) as the excitation source 14. In such an embodiment of the system the scan head 20 is replaced with a suitable barrier filter to define an excitation wavelength. Under either set of conditions, the excitation source 14 is capable of stimulating the specimen 10 with light across a range of the electromagnetic spectrum to stimulate the desired emissions. For example, ultraviolet, visible light and infrared are usable in the present system although wavelengths between 400 and 488 nm are preferable to stimulate the desired emissions without the potential to cause photochemical damage or difficulties in delivery due to opacity of the tissue at wavelengths shorter than 400 nm.

A second option is the use of red to infrared light at a wavelength between 690 nm and 900 nm delivered in short pulses, for example picosecond or nanosecond, to elicit emissions via multiphoton excitation. This wavelength range overcomes drawbacks associated with the other wavelengths. While many manufactures produce laser systems which could be modified to serve the present invention, an exemplary ophthalmoscope includes confocal scanning laser ophthalmoscopes such as Rodenstock SLO 101 (available from Ottobnunn-Riemerling, Germany). Modifications of such devices within the ability of artisans include optionally replacing standard laser sources with light sources such as Spectra-Physics Tsunami titanium-sapphire pulsed lasers which are known to have appropriate pulse rates to excite multi-photon elicited fluorescence.

The light 16 impacts the specimen 10 in an identifiable target area that, when stimulated, produces an auto-fluorescent emission in response. As more fully discussed below, the area of the eye known as Bruch's membrane fluoresces at a wavelength between 410 nm and 530 nm. For detection of this autofluorescence one of the following bandpass filters 34a (nm/±bandwidth) of 450/20, 450/40, 470/20, 470/40, 490/20 and 490/40 is placed between a beamsplitter 32 and one detector 36a. These filters are considered optimal for detection of autofluorescence emitted by these structures. Another layer, known as the retinal pigmented epithelium (RPE) is anchored to the Bruch's membrane. Lipofuscin granules within the RPE fluoresce when excited at the same wavelengths but will emit light at a wavelength between 505 nm and 700 nm. For detection of this autofluorescence one of the following bandpass filters 34b (nm/±bandwidth) of 525/20, 555/20, 620/20, or a 620 longpass filters is placed between 32 and the detector 36b. These filters are considered optimal for detection of autofluorescence arising from lipofuscin granules within the RPE. These auto-fluorescent emissions, generally indicated by the numeral 30, are passed in the present example through a beam splitter 32 to allow collection of the several emission wavelengths. To assist in limiting detection to the most meaningful wavelengths, the split emissions 30ₐ, 30_{b} are passed through respective filters 34ₐ, 34_{b} (as discussed above) before entering detectors 36ₐ, 36_{b}. In the present example, filter 34ₐ passes a range associated with Bruch's membrane fluorescence and filter 34_{b} passes a range associated with Lipofuscin fluorescence. Such filters are known and commercially available from Omega Optical, Brattleboro, Vt. Additionally, those skilled in the art will appreciate that the detector may be a photodiodes, photomultipliers, video cameras, CCD cameras, and the like, may alternately be incorporated to optimally receive emissions at selected wavelengths.

The detectors 36, in turn, are connected to the processor 12 so that the data 38 indicative of the florescence can be processed. Processor 12, incorporated within the ophthalmoscope or external thereto, receives data from detector 36ₐ indicative of the auto-fluorescent emission 30ₐ associated with Bruch's membrane through an input. The processor 12 also receives data from detector 36_{b} indicative of the auto-fluorescent emission 30_{b} associated with the RPE associated lipofuscin. The data received includes amplitude, wavelength, scanned, positions such as XY, Xt, XYZ, and the like. In one embodiment, the processor 12 receives an amplitude associated with the Bruch's membrane fluorescence 30ₐ and calculates a ratio between the amplitude of the RPE associated lipofuscin fluorescence 30_{b}. Desirably, the processor 12 compares autofluorescence emissions 30 within the same data set, that is, within the same specimen. This minimizes inaccuracies due to comparisons between standardized data sets taken from or averaged over a large sample. In another words, disease of a specimen is indicated by a comparison of data sampled and compared to data taken from the specimen itself. As will be more fully discussed below, macular degeneration is indicated when the ratio in regions of the macula exceeds that observed elsewhere in the fundus.

In another embodiment, the processor 12 plots the emission intensities to XY coordinates using a traditional reflected light image of the fundus as a reference. Then the intensities are compared to predetermined data thresholds derived from a control data set both spatially and quantitatively. This data then is used to form an image to graphically display intensity variations between target areas and thus regions where pathologies are occurring that are not visible in the traditional fundus image alone.

In order to overcome the difficulties and dangers of using UV illumination for fundus autofluorescence, one iteration of the present invention proposes an ophthalmoscope that scans the retina using a pulsed infrared laser capable of multi-photon excitation to produce emissions from Bruch's membrane, drusen and RPE/lipofuscin. This laser scanning technology produces molecular excitation in a target material by simultaneous absorption of two or more photons (multi-photon). Multi-photon excitation provides a unique opportunity to excite molecules normally excitable in the UV range with infrared (IR) or near-IR light. The advantages of using longer wavelengths, near-IR or IR light, are possibly less photodamaging to living cells and conveniently available solid state picosecond and femtosecond laser sources. In practice, the configuration of multi-photon laser scanning microscopy can be identical to the existing single photon systems. The data obtained is processed to produce a ratio of fluorescence intensities among those spectra elicited as well as images that can be used for measurements of retinal features such as the thickness of Bruch's membrane. This ratio of intensities of the different fluorescent peaks elicited are then used as the diagnostic/prognostic criteria for the detection of retinal diseases.

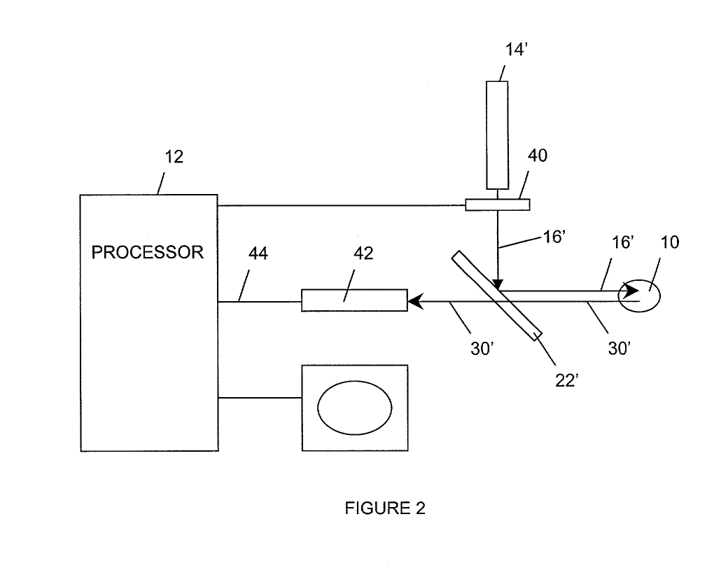

With reference now to FIG. 2, an alternate embodiment of a system which suitably practices the invention is provided where like components are identified by like reference numerals. A specimen 10, such as an eye, is placed relative to the system under control of a processor 12. The processor 12 controls an excitation source 14′ such as a laser or an arc lamp which generates light 16′. In the arc lamp system, the light 16′ is passed through a narrow bandpass filter 40 which defines the wavelength of light to be used for excitation of fluorescence. As in FIG. 1, when a laser is used 40 is substituted with a scanhead. The light 16′ leaving the filter 40 is directed into other optics 22′ and is focussed on the specimen 10. Those skilled in the art will appreciate that while the optics 22′ are simply illustrated, actual optics associated with a system for fluorescence imaging are considerably more complex. In the arc lamp excitation source of the present example, the excitation source 14′ and filter 40 combination provide light at wavelengths between 400 and 488 nm. This range suitably elicits emissions without little potential to cause photochemical damage or other difficulties in delivery due to the opacity of tissue at wavelengths shorter than 400 nm. Commercially available arc lamps are available from companies such as Oriel Instuments (Stratford, Conn., 05515, USA).

The excitation light 16′ is focussed on the specimen 10 and auto-fluorescent emissions 30′ are generated. In the embodiment illustrated in FIG. 2, emissions 30′ are received in a multiple wavelength detector apparatus 42 In the multiple wavelength detector apparatus 42 the emission beam 30′ is split in front of the camera and is focussed on the CCD chip resulting in two images side by side on the same chip. Advantageously with this detector arrangement, differences induced by varying detector sensitivity, beam splitter misalignment and the like are eliminated. Suitable multiple wavelength detectors are commercially available from Optical Insights of Santa Fe, N. Mex. under the name MultiSpec. Data 44 from the multiple wavelength detector 42 is provided to the processor 12 for ratiometric calculations.

### EXAMPLE

The example below was conducted using sections of donor eye tissue.

In order to examine the auto-fluorescent emissions of tissue with respect to its origin, 8 μm sections derived from maculae of unfixed posterior poles were prepared and three sections from each eye were examined by confocal microscopy using light at 633 nm, 568 nm, 488 nm, and 364 nm for excitation. Specimens were taken from tissue obtained from donor eyes of elderly persons including those free from AMD and those with AMD. XY-λ datasets were accumulated for emitted light in 10 nm increments from 400-800 nm. Since no emissions were excited at wavelengths shorter than the excitation wavelength, no data are presented for these regions of the spectra. In addition, due to reflectance at the excitation wavelength (λₑₓ) the reflection peak is omitted from all data sets presented where λₑₓ≦488 nm.

Referring now to FIG. 3, representative images are shown for a control specimen, generally indicated by reference numeral 50A, and a diseased AMD specimen, generally indicated by reference numeral 50B. Attention is drawn to the hard drusen deposit 52 illustrated in the control specimen 50A. More variability is present in the AMD specimen 50B. While all diseased specimens did not necessarily include hard drusen deposits, all did contain some form of deposit between the RPE and Bruch's membrane. The most common finding was basal laminar deposits. Basal laminar deposits were absent from all fields in control specimens.

Spectral scans were performed starting with excitation wavelength, λₑₓ of 633 nm and moved to progressively shorter wavelengths to minimize any potential for photobleaching. The effects of photobleaching were to lower the average intensity of emission in a given field equivalent to raising the baseline by ˜10% in rescanned sections. The emission peak (λₘₐₓ) values for Bruch's membrane, drusen, and lipofuscin at each excitation wavelength are reported in table 1.

TABLE 1
λₘₐₓ values* for Bruch's membrane, drusen, and RPE.

| | | Bruch's | | |
|---|---|---|---|---|
| λ_{Ex.} (nm) | Speciman | Membrane | Drusen | RPE |
| 364 | Control | 485 | 485 | 555 |
| | AMD | 485 | 490 | 540 |
| 488 | Control | 540 | 545 | 555 |
| | AMD | 540 | 545 | 555 |
| 568 | Control | 610 | 610 | 615 |
| | AMD | 600 | 605 | 615 |
| 633 | Control | 645 | 645 | 655 |
| | AMD | 650 | 650 | 655 |

*λₘₐₓ values are ± 5 nm. Data are presented as Mean (n = 3).

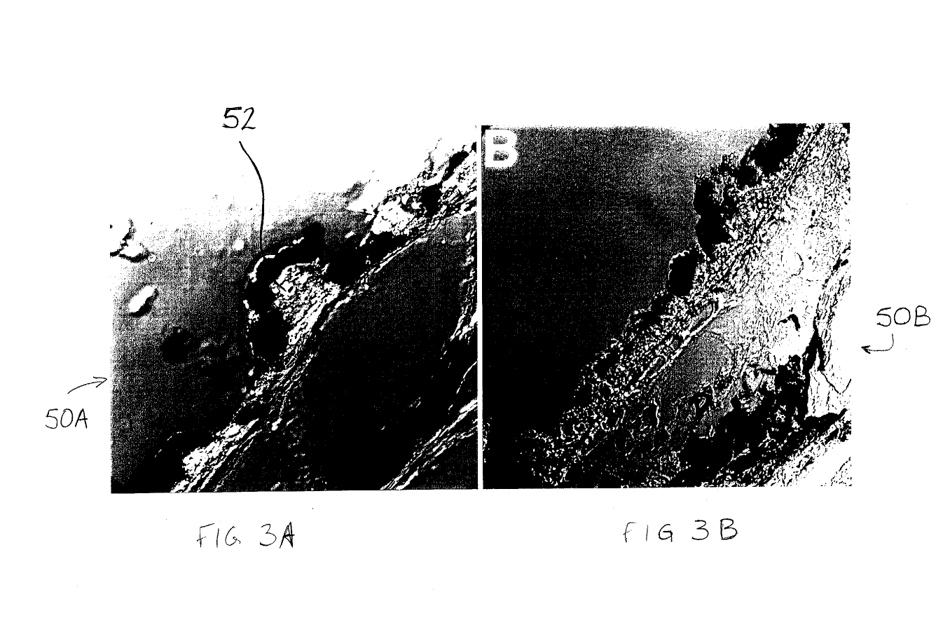

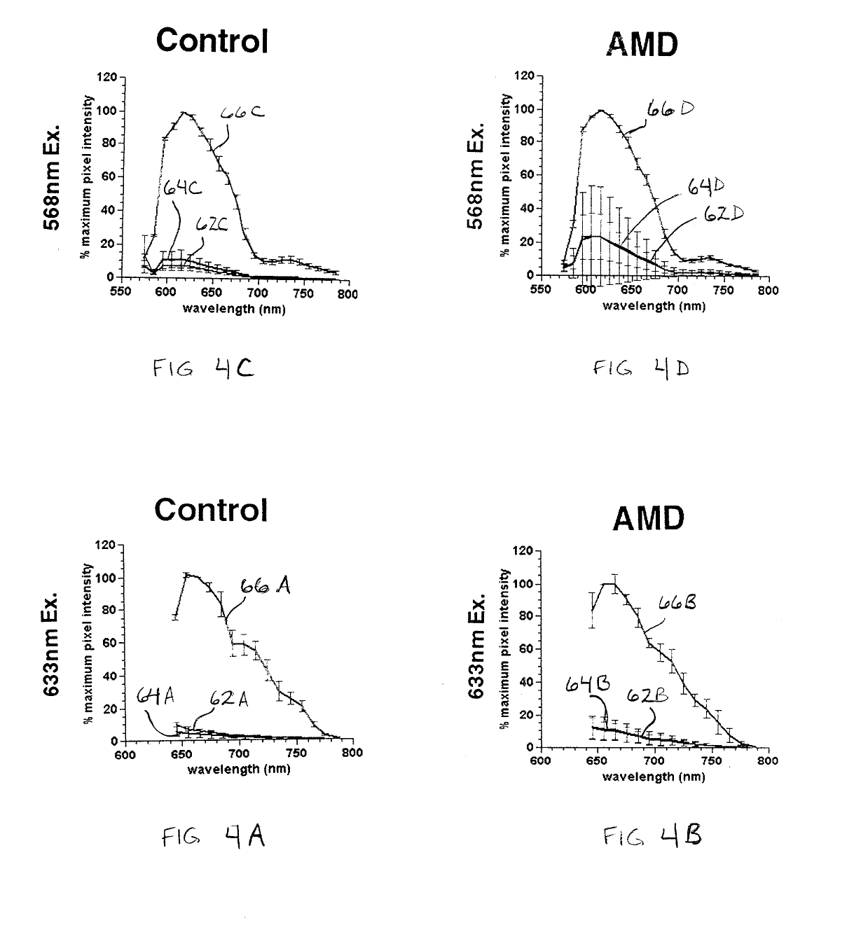

Referring now to FIGS. 4A-4D, at the excitation wavelength, λₑₓ of 633 nm and 568 nm in both control (FIGS. 4A and 4B) and AMD samples (FIGS. 4C and 4D), very small differences were noted in the emission peak λₘₐₓ values associated with each of the regions examined. These differences were confined to slight blue shifts of drusen 62 and Bruch's membrane 64 relative to lipofuscin 66, though in effect they all had the same spectrum. RPE associated lipofuscin 66 was the dominant signal. While no significant difference was noted in the spectra or intensities, an increase in the intensities of both Bruch's membrane and drusen was noted, however, in AMD eyes though this difference failed to show significance.

Referring now to FIGS. 5A and 5B, at excitation wavelength λₑₓ of 488 nm, a 10 nm difference was reproducibly obtained between the spectrum of Bruch's membrane 74 and drusen 72 vs. lipofuscin 76. The difference in emission peak λₘₐₓ (see Table 1) was identical in both control and AMD eyes. Lipofuscin 76 was however the dominant fluorophore in both control and AMD eyes (see Table 2). Interestingly, a significant increase in the intensity of Bruch's membrane 74 fluorescence was detected in the AMD eyes when compared to lipofuscin 76 fluorescence. In table 2, the percent of mean maximum pixel intensity is shown for the emission peak λₘₐₓ values in table 1. This measure is relative to the most intense finding in the section series which is assigned the value of 100%. All other values are relative to the pixel intensity in this region. This is a useful examination of intensity at the emission peak λₘₐₓ value of each spectrum and indicates the strongest signal and reproducibility of that signal intensity for each region and spectrum. A second useful analysis has been to examine the ratio of peak intensities normalizing against the intensity of lipofuscin 76. In control eyes Bruch's membrane 74 fluorescence at emission peak λₘₐₓ was 56±14% (Mean±SEM) of the intensity of lipofuscin 76, whereas in AMD eyes Bruch's membrane 74 fluorescence was 101±27% (Mean±SEM, P≦0.031). However, no difference was detected in the intensity of drusen 72 with respect to lipofuscin 76 in control (59±19%, Mean±SEM) vs. AMD eyes (58±5%, mean±SEM).

TABLE 2
Percent maximum pixel intensities* of Bruch's
membrane, drusen, and RPE.

| | | Bruch's | | |
|---|---|---|---|---|
| λ_{Ex.} (nm) | Speciman | Membrane | Drusen | RPE |
| 364 | Control | 82 ± 9 | 77 ± 4 | 96 ± 2 |
| | AMD | 91 ± 9 | 64 ± 12 | 62 ± 7 |
| 488 | Control | 53 ± 13 | 57 ± 17 | 97 ± 2 |
| | AMD | 81 ± 14 | 49 ± 6 | 85 ± 9 |
| 568 | Control | 31 ± 10 | 21 ± 7 | 100 |
| | AMD | 45 ± 9 | 33 ± 4 | 100 |
| 633 | Control | 9 ± 1 | 11 | 100 |
| | AMD | 15 ± 3 | 15 ± 4 | 100 |

*Data are Mean ± SEM (n = 3).

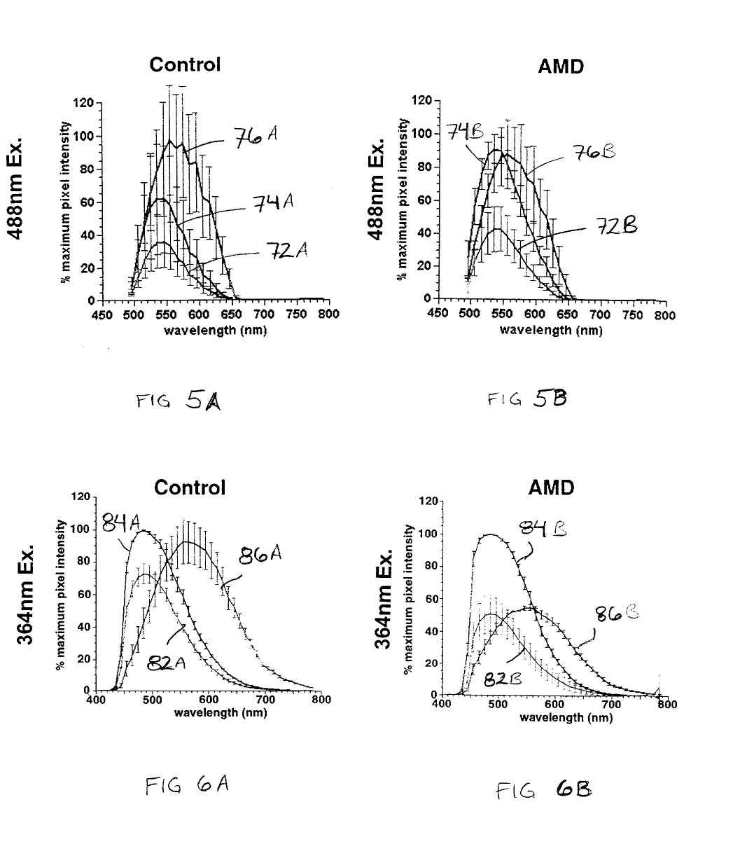

With reference now to FIGS. 6A and 6B, at excitation wavelength λₑₓ=364 nm, a substantial difference was found between in emission peak λₘₐₓ (see Table 1) for Bruch's membrane 84 and drusen 82 with respect to lipofuscin 86. In both control and AMD eyes, Bruch's membrane 84 exhibited a emission peak λₘₐₓ value of 485±5 nm, similar to the emission peak λₘₐₓ obtained for drusen 82 (Table 1). However lipofuscin 86 had a emission peak λₘₐₓ of 555±5 nm in control and 540±5 nm in AMD eyes. Thus, we could clearly delineate different spectra for Bruch's membrane 84 and drusen 82 with respect to lipofuscin 86. Interestingly in AMD eyes Bruch's membrane 84B became the dominant fluorophore (see table 2). Furthermore a substantial difference was found in the intensity of Bruch's membrane 84 fluorescence with respect to lipofuscin 86 in AMD eyes vs. controls. This difference; Bruch's membrane was 86±11% (Mean±SEM) of lipofuscin intensity in control eyes vs. 154±29 (Mean±SEM) in AMD eyes was significant at P≦0.024. A small difference was noted for drusen as well with drusen being 80±4% (Mean±SEM) of the intensity of lipofuscin in control eyes and 102±7% (Mean±SEM) in AMD eyes, though this difference was not significant.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated by reference hereto for the purpose of describing and disclosing the techniques and methodologies which are reported in the publications which might be used in connection with the invention. Nothing herein is to be construed as an admission that the invention is not entitled to antedate such disclosure by virtue of prior invention.

There have been described and illustrated herein embodiments of the apparatus and method of using the same to diagnose and prognosticate retinal diseases. While particular embodiments of the invention have been described, it is not intended that the invention be limited thereto. It is intended that the invention be as broad in scope as the art will allow and that the specification be read likewise. For example, those skilled in the art will appreciate that certain features of one embodiment may be combined with features of another embodiment to provide yet additional embodiments. It will therefore be appreciated by those skilled in the art that other modifications could be made to the provided invention without deviating from its spirit and scope as so claimed and described.

Having thus set forth the preferred embodiments, the invention is claimed to be:

1. A method comprising:
receiving a first auto-fluorescent emission from an identifiable area of a specimen, the first auto-fluorescent emission comprising a first range of wavelengths;
receiving a second auto-fluorescent emission from the identifiable area of the specimen, the second auto-fluorescent emission comprising a second range of wavelengths; and
comparing characteristics of the first and second auto-fluorescent emissions, where selected comparisons indicate disease of the specimen.

2. The method as set forth in claim 1, further comprising:
stimulating the specimen with light.

3. The method as set forth in claim 2, where the light comprises a wavelength in the range of 400 nm and 490 nm.

4. The method as set forth in claim 2, where the light comprises pulsed laser light comprising a wavelength in the range of 690 nm and 900 nm capable of eliciting autofluorescence via multi-photon excitation.

5. The method as set forth in claim 1, where the receiving a first auto-fluorescent emission occurs simultaneously with the receiving a second auto-fluorescent emission.

6. The method as set forth in claim 1, where the first predefined range of wavelengths ranges between 410 nm and 530 nm.

7. The method as set forth in claim 1, where the second predefined range of wavelengths ranges between 505 nm and 700 nm.

8. In a system for evaluating a specimen including an excitation source for exciting the specimen, a detector for detecting specific fluorescence from the specimen in response to the excitation, and a processor for processing data received from the detector, the processor comprising:
a first input in data communication with a first detector element providing data indicative of a first fluorescence in a first wavelength band from an identifiable area of the specimen;
a second input in data communication with a second detector providing data indicative of a second fluorescence in a second wavelength band from the identifiable area of the specimen; and
a comparator that compares the data indicative of the first and second fluorescence, where selected comparisons indicate disease of the specimen.

9. A method for diagnosing retinal characteristics, said method comprising:
emitting light of a predetermined excitation wavelength into a target area of an eye;
detecting a first autofluorescence from said target area of said eye in response to the emitting;
detecting a second autofluorescence from said target area of said eye in response to the emitting;
calculating a ratio of an intensity of said first autofluorescence and an intensity of said second autofluorescence; and
comparing said ratio to a predetermined data set to identify a retinal characteristic.

10. The method of claim 9, wherein said light comprises infrared pulsed laser light.capable of eliciting fluorescence via multiphoton excitation.

11. The method of claim 9, wherein said light comprises filtered light derived from a source of white light.

12. The method of claim 9, wherein said excitation wavelength is between 690 and 900 nm.

13. The method of claim 9, wherein said excitation wavelength is in the range of 400 nm and 490 nm.

14. The method of claim 9 wherein said target area is located in a posterior region of said eye.

15. The method of claim 9, wherein said characteristic is age-related macular degeneration (AMD).

16. An optical scanning spectroscopic apparatus comprising:
a light source to direct light into a posterior region of an eye;
means for detecting autofluorescence from the posterior region of the eye, said autofluorescence comprising a plurality of detectably distinct wavelengths responsive to the directed light; and
means for processing the plurality of detectably distinct wavelengths and comparing said plurality of detectably distinct wavelengths to a control data set.

17. The apparatus in claim 16 wherein said light source comprises a laser capable of eliciting fluorescence by multi-photon excitation.

18. The apparatus in claim 16 wherein said light source comprises intense light with a wavelength between 400 and 490 nm.

19. The apparatus in claim 16 wherein said means for detecting permits simultaneous detection of autofluorescence at two wavelengths.

20. The apparatus in claim 16 further comprising a filter disposed between the light source and the eye where the filter substantially blocks light around a defined band of wavelengths.
